(19) United States
(12) Patent Application Publication
(10) Pub. No.: US 2010/0049055 A1
(43) Pub. Date: Feb. 25, 2010

(54) Method and apparatus for visual characterization of tissue

(75) Inventors: Thomas Freudenberg, Berlin (DE); Karl-Heinz Schönborn, Berlin (DE)

Correspondence Address: THE WEBB LAW FIRM, P.C., 700 KOPPERS BUILDING, 436 SEVENTH AVENUE, PITTSBURGH, PA, 15219, US

(73) Assignee: W.O.M. WORLD OF MEDICINE AG, Ludwigsstadt (DE)

(21) Appl. No.: 11/921,374

(22) PCT Filed: May 31, 2006
(86) PCT No.: PCT/DE2006/000941

§ 371 Date: Oct. 9, 2008

(30) Foreign Application Priority Data
May 31, 2005 (DE) 10 2005 025 241.9
May 31, 2005 (DE) 10 2005 025 242.7
May 31, 2005 (DE) 10 2005 025 243.5

(51) Int. Cl. A61B 6/00; G01N 21/64

(52) U.S. Cl. 600/475; 356/402; 600/407; 600/477

(57) ABSTRACT

A method for visual characterization of human or animal tissue formed from cells, having the following steps: a radiation source for emitting directional electromagnetic radiation is provided; —the tissue to be irradiated with the radiation, this producing a reflection in the tissue which is characteristic of the tissue, with—the radiation which has penetrated the tissue having, within an excitation region extending transversely with respect to the direction of propagation of the radiation, a sufficient intensity to excite a reflection in the tissue. The radiation emitted by the radiation source is impressed with an intensity profile, transversely with respect to its direction of propagation, which is such that the excitation region covers a plurality of cells of the tissue and the excited reflection originates from inter-cell tissue properties. The invention relates to a method in which the radiation emitted by the radiation source is periodically deflected transversely with respect to the direction of propagation such that the radiation periodically scans a region of the tissue around the measurement point which extends over a plurality of cells of the tissue. The invention also relates to apparatuses for carrying out the methods.

The invention relates to a method for visual characterization of human or animal tissue formed from cells, and to an apparatus for carrying out the method.

Epidermal, cutaneous, adnexal and mucosal changes were predominantly diagnosed clinically until the end of the 20th century. Since the 1980s, a series of imaging methods have been able to become established in dermatology as a result of the rapid development of technology, primarily of computing technology.

In this case, fluorescence-based tumor diagnosis is of special relevance. A number of devices based on linear UV excitation by means of a xenon lamp are already commercially available here. In general, the autofluorescence intensity is evaluated in characteristic wavelength ranges (Storz, Richard-Wolf GmbH, Olympus) against a white light background image.

In particular, the working group of H. van den Bergh investigates how a considerable improvement in diagnosis can be achieved by a sequentially multicolored excitation, lifetime resolution and special data evaluation [H. van den Bergh Med. Laser Appl. 18 (2003)1, 20 and citations therein]. In all cases the lateral resolution lies in the sub-millimeter range. A differentiation in the depth is not possible for fundamental reasons.

The aim of realizing a three-dimensional fluorescence representation of tissue with acceptable luminous efficiencies came closer to being achieved only with the advent of multiphoton fluorescence microscopy (W. Denk, J. H. Strickler, W. W. Webb, “2-Photon laser scanning fluorescence microscopy,” Science 248 (1990), 73-76). In this method, the spatially resolved fluorescence detection of confocal microscopy, which causes very low fluorescence signals and thus low scanning rates, is replaced by localization by means of multiphoton excitation. Moreover, in this method it is possible to use wavelengths in the so-called optical window of tissue (700 nm-1000 nm) for the excitation. The focus of research in this field since work began has been on the spatially resolved representation of the concentration of various biomolecules in the cellular and subcellular range (overview W. R. Zipfel et al. Nature Biotechnology 21 (2003), 11, 1369-1377 and citations therein).

The field of multiphoton microscopy (tomography) also encompasses current work by the group of Prof. Karsten König (K. König et at. J. Biomed. Opt. 8 (2003) 3, 432-439). The intracellular structures represented with a spatial resolution in the submicron range permit statements with regard to cell division and metabolism behavior of individual cells and thus therapy control, but not the examination of extensive tissue areas. The diagnostic approaches based on multiphoton microscopy proceed from the interpretation of the intracellular metabolism processes and the cellular structure.

For the relevant spatially resolved representation on the basis of focused fluorescence excitation, reference may be made to the long established confocal fluorescence microscopy as prior art. It yields horizontal sectional images with a resolution in the range of the wavelength used. The advantages of two-photon excitation in this method, namely by excitation in the “optical window”, and significantly better spatial resolution even in scattering tissue, have been discussed and demonstrated [Review: W. R. Zipfel, R. M. Williams, W. W. Webb, nature biotechnology, 21/11, November 2003]. By way of example, mention shall be made here of the work by Levene et al. [M. J. Levene et al. In vivo multiphoton microscopy of deep brain tissue. J Neurophysiol 91: 1908-1912, 2004] and Theer et al. [P. Theer et al., Two-Photon imaging to a depth of 1000 mm in living brain tissue . . . . Optics Lett. VOL. 28(12), 2003] in which nerve cells and blood vessels of rats and mice were able to be represented in vivo to a depth of 1 mm. This work proves that confocally nonlinearly excited fluorescence can be detected as spatially resolved information even from a large depth in the tissue. However, with regard to tumor diagnosis these methods have been able hitherto only to obtain morphological and intercellular information, and no statements about the metabolism state of tissue regions. The important information for therapeutic decisions, such as the spread and overall activity of a tumor, has not been able to be supplied hitherto.

The method of two-photon microscopy and later of multiphoton microscopy was made public by the documents U.S. Pat. No. 5,034,613 and U.S. Pat. No. 6,166,385 and accompanying publication. The concept of these methods is based on the fact that the three-dimensional spatial resolution is achieved by the narrow spatial delimitation of a fluorescence excited by two- or multiphoton processes. The obtaining of the image itself is based on the raster scanning of the examination region with the excitation radiation.

The resolution of two- or multiphoton processes is linked to the simultaneous absorption of two or more photons in the excitation region. Therefore, high intensities, such as are provided by pulsed laser systems, are generally required for this. If the average power of the laser has to be limited in order to avoid tissue damage in the excitation region, these are typically picosecond or femtosecond pulses or sequences of such pulses.

The two- or multiphoton absorption inherent to the method also governs a corresponding intensity dependence of the process: in the case of a two-photon process, the yield is typically dependent quadratically on the intensity, and in the case of a three-photon process on the third power of the intensity, etc.

The three-dimensional spatial resolution of the method for which two- and multiphoton microscopy are mentioned here by way of example is based on two interlinked effects which are described on the basis of the laser systems typically used:

In the image plane of the optical system which focuses the excitation radiation into the target material, the delimitation results from the properties of the radiation source and of the optical system used. This is described by the radius of the beam waist w₀, which, for the common case of fundamental mode radiation, to which reference is made here as an example without restricting the generality of the concept of the invention, is calculated approximately as follows:

$${w}_{0} \approx \frac{2 λ \cdot f}{π \cdot D}$$

In this case, λ is the wavelength of the radiation, f is the focal length of the optical system used, and D is the illuminated diameter when radiation enters into the optical system. As can easily be discerned, the diameter of the excitation region in the image plane of the optical system can be set for example by choice of the ratio f/D.

Along the propagation direction of the radiation (z direction), the radius w(z) of the fundamental mode bundle—considered here by way of example again—increases:

$$w ( z ) = {w}_{0} \cdot \sqrt{1 + {( \frac{z \cdot λ}{π \cdot {w}_{0}^{2}} )}^{2}}$$

As the radius of the bundle increases, the intensity of the excitation radiation and hence the yield of the process decrease.

$$I ( z ) = \frac{{I}_{0}}{1 + {( \frac{z \cdot λ}{π \cdot {w}_{0}^{2}} )}^{2}}$$

$$Rayleigh length {z}_{0} = \frac{π \cdot {w}_{0}^{2}}{λ}$$

In this case, I₀ is the intensity at the focus of the excitation. This fact is characterized by the so-called Rayleigh length, which specifies that distance from the focus at which the intensity has decreased by half.

In accordance with the abovementioned quadratic or higher relationship between the yield of the process and the excitation intensity, it becomes clear that the excitation region is also delimited along the propagation direction, which is called the z axis here.

The description of these relationships also makes it clear that in this arrangement it is not possible to define the dimensions of the excitation region independently of one another by the choice of excitation radiation and optical system. The extent of the excitation region in the z direction increases quadratically with the radius of the excitation beam at the focus.

On account of this fact, for multiphoton microscopy, for example, it is not possible to carry out overview measurements with a reduced resolution—that is to say enlarged focus—and a correspondingly increased yield without resolution in the propagation direction of the excitation radiation thereby being lost more than proportionally. This fact is disadvantageous particularly when the intensity of the excitation radiation is limited in order to avoid undesirable processes in the target material.

The following should furthermore be taken into consideration in the case of the targeted nonlinear excitation of processes in organic or inorganic substances:

The spot in which nonlinear processes proceed is shaped as a waist of a Gaussian beam and is comparatively elongated in the case of apertures that are not excessively large. What is desired for the evaluation, however, is usually a compact form of the excitation volume, that is to say one in which the diameter of said volume and the extent perpendicular to the beam axis have approximately the same order of magnitude.

In this case, in principle, as in the methods mentioned, the three-dimensional image is usually generated by scanning. The restriction described in the aforementioned paragraph is eliminated by additional, inventively novel methods and apparatus equipment which are disclosed below.

A further limitation of the previous methodologies which restricts the use of the nonlinear fluorescence methodologies outside clinical and biological research is the small volume of the methods in accordance with the prior art is the small extent of the tissue volume examined. This is of the order of magnitude of laterally 500×500 μm and in terms of the depth 200 to 400 μm. The limitation is limited by the excessively high spatial resolution, on the one hand, as already described. On the other hand, the data quantities and rates which in the case of a real-time evaluation—such as is necessary for use for clinical diagnosis and early identification of diseases, in particular of a tumorous nature—also limit the extremely desirable clinical use of the methodology.

The present invention is based on the problem of specifying a method and an apparatus for visual characterization of tissue which permit a more reliable differentiation between healthy and diseased tissue than has been possible hitherto.

The aforementioned problem is solved by means of the method having the features of Claim 1, by means of the method having the features of Claim 33, by means of the apparatus in accordance with Claim 46 and also the apparatus in accordance with Claim 61. Particularly preferred embodiments of the invention are specified in the subclaims.

Accordingly, a method for visual characterization of human or animal tissue formed from cells is specified, which method has the following steps:
providing a radiation source for emitting directional electromagnetic radiation;
irradiating the tissue to be characterized with the radiation, whereby a reflection radiation which is characteristic of the tissue is generated in the issue,
wherein the radiation that has penetrated into the tissue has, within an excitation region extending transversely with respect to the propagation direction of said radiation, a sufficient intensity to excite a characteristic reflection radiation in the tissue, wherein
the radiation emitted by the radiation source has impressed on it an intensity profile transversely with respect to its propagation direction, said intensity profile being such that the excitation region covers a plurality of cells of the tissue and the excited reflection radiation originates from inter-cell tissue properties.

The method according to the invention provides the basis for an innovative fluorescence-diagnostic, imaging, non-invasive tumor diagnosis method which is suitable for early identification and for screening (in vivo and in vitro). The method supplies the characterization of the tissue or metabolism state and hence of diseased, in particular tumorous, changes with a meaningfulness and spatial resolution which are oriented to the clinical objective and are not currently available to the physician. The tissue state is determined from the spectroscopic data with supracellular averaging and is represented to the physician in a morphological sectional image which encompasses a number of millimeters and permits an assessment of tumors with regard to their extent, position and aggressiveness.

The patient benefits primarily from the improved early identification and diagnosis. These make it possible to avoid operative interventions or—in the case of the localization of early stages—to significantly reduce them by minimally invasive methods. Health insurers save treatment costs as a result of costs reduced by two thirds in comparison with the hitherto unavoidable biopsies and also as a result of a significant reduction in the number and severity of the operative interventions. Insofar as surgical interventions cannot be avoided or replaced by minimally invasive methods, such as PDT (photodynamic therapy), the severity of the intervention and its healing-related and aesthetic effects are significantly reduced because the demarcation between healthy and diseased tissue can be made significantly more exactly than hitherto.

However, the novel method can also advantageously be used for other measurement and characterization tasks e.g. in the field of pathology, biochemistry, biotechnology, for living or non-vital organic and for inorganic substances in a solid or liquid state. To afford a better understanding, all the specifics are presented below using the example of the fluorescence diagnosis of tumors, without thereby wishing to restrict the general validity and scope of the concept of the invention.

In addition to the field of application presented first, in the area of skin, further areas of application in the area of accessible body cavities (ENT, gynecology) and in endoscopically accessible internal parts of the body (gastroenterologyr urology, bronchoscopy) are part of the field of application of the invention.

In the treatment of malignant diseases, early identification, alongside the early stages of cancer, primarily of the preliminary stages, is of crucial importance for the prospects of recovery. Moreover, the incidence of such preliminary stages of cancer is rising as a result of increasing life expectancy, which is also intensified by lifestyle habits such as e.g. intensive insolation, diseases caused by sexually transmitted viruses and environmental noxae. The following shall be mentioned here as examples:
a) actinic keratosis, a disease triggered by additive UV exposure, which can turn into a skin cancer, basal cell carcinoma, and
b) the genital warts triggered by HPV viruses, “condylomata”, which are not only responsible for triggering cervical cancer in women but can also induce cancers of the anogenital region in men and women.

While a manifest cancer disease can often be clearly identified (“diagnosis on sight” in approximately 50% of the diseases), its preliminary stages and very early stages are often inconspicuous in visual diagnosis. This is one reason why screening examinations do not produce the desired effect. They can either be “false negative”, that is to say do not identify an existing cancer disease in good time and therefore miss the chances for a cure by means of minimally invasive treatments. Equally unfavorable are “false positive” diagnoses, which, besides causing a considerable psychological burden on patients, until a cancer diagnosis has been ruled out, also entail many complicated examinations which not only place an additional burden on and endanger the patients but also cause considerable resultant costs.

On the other hand, minimally invasive methods such as cryotherapy and photodynamic therapy and recently also drug treatment (active ingredient Imiquimod) are becoming more and more important in the treatment of epithelial tumors. However, since these treatment methods work without excision, they lack the possibility of histological examination used not only to ensure the diagnosis but also to obtain information about the aggressiveness of the tumor (“grading”) and about transgressing of the boundary structures and penetration into adjacent tissue (‘staging’). Therefore, prerequisites for screening include not only unambiguous diagnosis by means of the spatial assignment of the fluorescence but also a statement about the spread and the degree of invasion.

It is evident from this that the method according to the invention advantageously meets the medical-diagnostic method requirements with regard to a spatially resolved diagnosis embedded into the morphological framework of the examination region whose dimensions, in accordance with clinical experience, should not be less than 4 mm laterally and 0.5 mm in terms of depth.

In one preferred development of the method, impressing the intensity profile consists in imaging the radiation into the tissue via a lens. In this case, the lens has a numerical aperture of approximately 0.3 to 1.5.

In a further preferred embodiment of the invention, impressing the intensity profile consists in splitting the radiation emitted by the radiation source into at least two partial beams, wherein the excitation region is defined by the position of the partial beams. In an advantageous manner, the partial beams are in each case focused into the tissue in such a way that the focal points for the partial beams are spaced apart from one another.

The basic concept of this development of the invention is that a plurality of partial beams which differ in terms of their direction are shaped from the excitation beam generated by the radiation source. By means of an optical focusing system, a bundle of foci arise in the focal plane thereof in accordance with the number and arrangement of the partial beams. If the distance between them in the focal plane is set in a suitable manner, then a delimitation of the multiphoton excitation region which is at least approximately independent of the number of partial beams results along the propagation direction of the excitation radiation.

It is particularly advantageous if the reflection radiation is a fluorescence signal, e.g. a two- or multiphoton fluorescence. The previous methods of clinical fluorescence diagnosis, of both auto- and xenofluorescence, only detect a summation image of superficial tissue layers. The excitation is effected by means of short-wave UV or blue light, wherein a “false negative” finding can be ascertained as a result of extinction by overlying non-metabolically active necrotic tissue, or, in the case of a tumor spreading under the healthy skin, the actual spread and hence the determination of the size of the tumor are estimated incorrectly. These disadvantages can be surmounted by narrow spatial delimitation of an NIR multiphoton excitation by means of lasers.

The methods of multiphoton microscopy developed in recent years utilize this procedure of excitation of fluophores in the tissue. They aim for high spatial resolutions (<1 μm) for the examination of cell-biological processes in subcellular dimensions. The methods of scanning microscopy which are used for this limit the scanning volume to field of view dimensions and scanning depths of the order of magnitude of a few hundred micrometers. Unlike in the method according to the invention, these values fundamentally cannot be extended to the abovementioned dimensions of the scanning volume which are required for clinical use.

Furthermore, the subcellular resolution of the conventional methods—in contrast to the supracellular resolution of the method according to the invention—does not permit the determination of the correlation between the fluorescence spectra of endogenous fluophores and the pathological states of corresponding cell regions. Multiphoton microscopy has matured into an effective instrument of cell-biological research. Clinical use for tumor staging is not its actual field of application.

In the area of tumor diagnosis, the method serves the purpose of drastically reducing the number of operative interventions actually required. First and foremost, skin tumors are at the focus of the treatment since they are readily accessible externally. Further reaching, in part even more significant applications result intra- and extracorporeally by means of an endoscopic and intraoperative applicator device with integrated scanning function.

Given corresponding extension and miniaturization provided according to the invention, there are far reaching future potentials for the invention in endoscopic diagnosis, too, primarily for gynecology and ENT medicine. In particular the differential diagnosis of cervical diseases and other diseases induced by human papilloma viruses, but also oral leukoplakia and the lichenoid preliminary stages of cancer are fields of application of the invention that should be taken very seriously.

For tumor staging and thus the indication with respect to minimally invasive methods, besides the size of the tumor the spreading depth and stromal invasion are of much more crucial importance. The previous sectional image methods, such as MRT, are not capable in this respect from the standpoint of their spatial resolution. High-resolution sonography at 100 MHz is beset by very many disturbing factors, if only owing to the fact that, as a contact method, it causes different thickness measurements at different sound head bearing pressures. Optical coherence tomography (OCT) could represent a solution for this on account of its high resolution. However, previous experience with all three methods has shown that a differentiation between swelling, inflammation and carcinogenic-pathological finding (“malignant/benign differentiation”) is not possible solely on the basis of morphological information.

Therefore, in one development of the method according to the invention, the fluorescence-spectroscopic, spatially resolved functional diagnosis is incorporated into a morphological representation of the examination region. For this purpose, the radiation emitted by the radiation source, for the additional characterization of the tissue by coherence reflectometry, is split into an excitation beam for exciting a fluorescence in the tissue and a reference beam, wherein the excitation beam is partly reflected back from the tissue and the reference beam is superimposed with the radiation reflected back from the tissue.

Particularly preferably, the intensity profile impressed on the radiation issuing from the radiation source has a characteristic variation having at least two intensity maxima. This “fingerprint” of the beam can be utilized to adjust the optical path lengths for the reference beam and the reflective beam. For this purpose, the optical path length for the reference beam is advantageously set in such a way that the intensity distribution impressed on the radiation emitted by the radiation source can be detected in the interference detector, wherein in this case the optical path length for the reference beam corresponds to the optical path length covered by the excitation beam from the radiation source to the focus and back to the interference detector.

If it is desired to combine multiphoton excitation processes with optical coherence reflectometry in order thus to obtain additional information about the excitation location, then the problem arises of the spatial assignment of the two processes or items of information. It consists, particularly in the case of optically inhomogeneous samples (e.g. tissue), in the fact that the inhomogeneous refractive index of the material influences the location of the multiphoton excitation and the signal of optical coherence reflectometry in different ways: the refractive power influences the path of the excitation light as far as the location of the excitation (focus) into the depth of the material only once. If the backscattered signals detects by the method of optical coherence reflectometry, then the refractive index influences the beam path thereof twice by virtue of the light having to penetrate through the material on the outgoing path and on the return path. Consequently, the spatial assignment to one another in particular along the propagation direction of the excitation radiation is lacking for the desirable combination of multiphoton excitation and optical coherence reflectometry. To summarize, for the combination of fluorescence spectroscopy and coherence spectroscopy there is, in particular, the preferred development
that the radiation of a laser suitable for excitation of two- or multiphoton processes and/or for optical coherence reflectometry is split into an excitation beam and a reference beam,
that the excitation beam has impressed on it transversely with respect to its propagation direction a structure such that an intensity structure having at least two maxima separated by one minimum is formed only in the focal plane of the downstream optical system or in the direct vicinity thereof transversely with respect to the propagation direction of the excitation light,
that this focal region corresponds to that region in which primarily the desired two- or multiphoton processes take place,
that the structured excitation beam is focused, by means of a suitable optical system, into the target that is to be excited and/or to be examined,
that the backscattered radiation arising in the target is captured by the optical focusing system and propagates oppositely to the original propagation direction of the excitation light,
that in this way a suitable optical deflection element is introduced without said backscattered radiation again passing through that element which has impressed on the excitation beam its structure transversely with respect to its propagation direction,
that the backscattered radiation, in an optically conjugate surface with respect to the focal plane, is brought to interference with the reference beam in such a way that the conjugate points with respect to the illuminated points of the target are completely detected by the reference beam,
that the interference pattern which arises there is detected and evaluated by means of a spatially resolving light-sensitive detector,
that the optical path length of the reference beam can be altered, and
that as indication for the correspondence of the location of the two- or multiphoton excitation to that location from which the backscattered optical signals originate, the image of the interference pattern is taken, reflecting that structure which occurs only in the focal region of the excitation radiation.

The basic concept of the invention consists in impressing on the excitation beam a structure which occurs only at the focus—that is to say at the location of the multiphoton excitation—or changes with the propagation length in such a way that a detectable change in said structure or more precisely its image occurs in the vicinity of the focus. The interference of the backscattered light in an optically conjugate plane with respect to the excitation plane will precisely will have an image of said structure. With a spatially resolving detection element for the interference signal, the reference beam path can then be adapted in terms of its length until the structure to be expected occurs in the interference pattern. This is the indicator for the superimposition of the location of the multiphoton excitation with that of the morphological scanning by means of optical coherence reflectometry.

To summarize, the following advantages, in particular, have been afforded:
the characteristically different fluorescence curves of tumorous and normal tissue permit tissue diagnosis with significant selectivity and specificity,
the tissue is scanned with a focus size expedient for the method in the supracellular range, thereby enabling a local assessment of the tissue metabolism in real time with an expedient resolution,
as an advantageous group of embodiments, the size of the scanning focus is decoupled from the size of the “scanning volume” to be assigned to a scanning point. The invention thereby eliminates unfavorable restrictions which, in the previously known optical diagnosis methods, obstructed the intended use striven for here.
By means of particular referencing methods, a local characteristic value for malignant/benign differentiation is determined (“spectral fingerprints”), which describes the metabolism status, the degree of new vessel formation or the structuring status of the tissue area under consideration largely independently of disturbing variables and enables an automatic prepathological assessment,
the combination of spatially resolved, functional biostatus assessment and morphological imaging represents a qualitative jump in the clinical diagnosis of the upper layers of skin,
in particular, a sufficient penetration depth as far as the basal membrane is achieved by means of choice of parameters and the specific evaluation method of the method, thereby enabling a differentiation between differently invasive forms of carcinogenic phenomena.

The radiation source is therefore used both for the multiphoton excitation and for optical coherence reflectometry. If appropriate, the spectral properties of the source should be configured in such a way that it enables the desired depth resolution (resolution along the radiation propagation). Said radiation is split into an excitation beam and a reference beam by means of a partly transmissive mirror. Both beams may additionally be provided with modulators which temporally modulate the intensity but not the optical path length.

In one particularly preferred variant of the method it is provided that
a) the tissue is irradiated with radiation in the wavelength range of 720-800 nm, whereby a two-photon fluorescence is excited in the tissue;
b) detection of the intensity of a first fluorescence signal at a wavelength of 460±30 nm and of a second fluorescence signal at a wavelength of 550±30 nm;
c) determination of the ratio of the intensities of the first and second fluorescence signals; and
d) a signal is generated in a manner dependent on the ratio determined in step c).

In this case, the wavelength range at 460±30 nm is significant for NAD(P)H and the wavelength range at 550±30 nm is significant for flavines. The formation of the ratio of the measured values from (a) and (b) serves for referencing the disturbing factors and obtaining an activity signal for the metabolic activity, which signal differs significantly between normal and pathological tissue volumes of the same type.

In a further preferred variant it is provided that
a) the tissue is irradiated with radiation in the wavelength range between 500-550 nm, and in this case
b) a first intensity of a two-photon fluorescence is detected at a wavelength of 340±40 nm (significant for tryptophan);
c) the tissue is irradiated with radiation in the wavelength range between 720-800 nm, and in this
d) a second intensity of a two-photon fluorescence is detected at a wavelength of 460±40 nm (significant for NAD(P)H);
e) the ratio of the first intensity determined in step b) and the second intensity determined in step d) is determined (for referencing the disturbing factors and obtaining a characteristic value that differs significantly between normal and pathological tissue volumes of the same type); and
f) generation of an electrical signal in a manner dependent on the ratio determined in step c).
g) An electrical signal is generated in a manner dependent on the ratio determined in step f).

A further preferred variant of the method provides the following steps:
excitation of two-photon fluorescence in the wavelength range 720-800 nm and a measurement of the fluorescence signal at 460±40 nm [significant for NAD(P)H] as a function of the tissue depth z take place,
storage of the measured values in a tissue area known to be healthy as a function of the depth (=“normal variation” as a function of z) and identical measurement in a suspicious area (=“suspicious variation” as a function of z),
wherein the two signal variations are subsequently related by ratio (=“indicator variation” as a function of z), and a limit of 20-40% deviation of the indicator from the value one in the suspicious area are used as an indication limit for an anomalous metabolism that differs significantly between normal and pathological tissue volumes of the same type.

Furthermore, the method can comprise:
irradiation and depth scan of a focused short-pulse laser beam in the wavelength range of 720-880 nm or 980-1080 nm and measurement of the returned radiation at exactly half the irradiation wavelength [indicator for type and structure of the collagen as structural feature] as a function of the tissue depth z in a in a tissue area known to be healthy as a function of the depth (=“normal variation” as a function of z), and
identical measurement in a suspicious area (=“suspicious variation” as a function of z), wherein
the two signal variations are subsequently related by ratio (=“indicator variation” as a function of z), and a limit of 30-50% deviation of the indicator from the value one in the suspicious area are used as an indication limit for an anomalous tissue structuring that differs significantly between normal and pathological tissue volumes of the same type.

In this case, the focused beam is moved through the tissue by means of a suitable (scanning) device. This movement can be performed in one of the following scanning modes
a) two-dimensionally in the sense of a horizontal (=a parallel to the tissue surface) scanning plane,
b) two-dimensionally in the sense of a vertical (=perpendicular to the tissue surface) scanning plane (“optical ultrasound”)
c) three-dimensionally in the sense of a scanning volume.

The aim, in particular, is finding tumors in the early stage (“carcinoma in situ”) and demarcation from further developed stages (migration of microvessels, break through of the basal membrane, loss of tissue structuring). Use is made of the following insights here:
a) Microcirculation is identified from the increased hemoglobin signal.
b) Resolution of the structure is identified from the locally deviating content of elastin and/or collagen.
c) The break through of the basal membrane is identified by the absence of the papilla structure by image evaluation.

In order to suppress disturbances and in order to compensate for influences of unknown scaling on the one hand of the measuring arrangement and on the other hand of the biological materials in the excitation and measurement beam path, referencing of the measurement signal is employed, in the case of which the measurement data of at least two significant substances are used, here in particular of the substance groups NAD(P)H, flavines and tryptophan.

As further significance-increasing information, use is made of morphological data, such as the local content of elastin or collagen, which can be detected by a dedicated fluorescence features elastin) or by nonlinear generation of a reference signal (preferably SHG=second harmonic generation=frequency doubling of the excitation radiation, preferably in the case of collagen).

A morphological image of the tissue area examined is shown to the physician on the monitor. Said image is based on the evaluation of the signals of the structure molecules (elastin and/or collagen) and the direct reflection of the excitation radiation (surface reflection and scattering amplitude).

A “tissue-spectroscopic fingerprint” is determined by the referencings mentioned and automatically assessed by the computer [at least two ratings (=“normal” or “deviating”] or three ratings (=“normal”, “deviating” or “unclear/indeterminate”)]. The regions of alternate metabolism are reproduced by false color representation in the monitor image.

### Apparatus Construction:

The apparatus comprises an operational apparatus with mains supply, control elements, integrated computer and connected monitor and also the handpiece with optical focusing system and scanner for the excitation radiation and the elements for collecting the measurement radiation (reflected and scattered beam at the excitation wavelength, SHG radiation at half the excitation wavelength and also the fluorescence radiation).

The excitation radiation is supplied by means of either fiber-optics, preferably as photonic fibers, or an articulated arm. In this case, elements of the supply optics for the fluorescence excitation radiation can also be used for collecting the measurement radiation.

As a particularly advantageous embodiment, in the case of the scanning methodology (“optical ultrasound”) fashioned analogously to imaging medical ultrasound, a scanning plane fixed in relation to the handpiece is placed vertically into the tissue and moved through the tissue by the physician in the manner that is customary in the case of ultrasound. The sectional image of the tissue with the additional information mentioned (“fingerprint” with automatic assessment) is displayed on the screen in real time.

In the case of operating states with high resolution where the manual positioning accuracy is insufficient or the unsteadiness caused by involuntary hand movement is too great for the method, the handpiece is fixedly emplaced and the displacement of the scanning plane is performed by electromotive or micromechanical actuating elements.

Preferably a laser, in particular a pulsed laser, is used as a radiation source. A possible excitation source which operates with pulses in the picoseconds range additionally makes it possible, through the use of a photonic fiber, also to generate wideband light for excitation in a plurality of colors. Such a light source has not been used hitherto either in fluorescence-based tumor diagnosis or in multiphoton microscopy.

The method is delimited from the known diagnosis methods by virtue of the fact that it assesses and represents the malignant/benign differentiation of the tissue state in a three-dimensional scanning image of the skin. In this case, the resolution should be chosen in such a way that—depending on the tissue depth under consideration—a number of approximately 20 to 200 000 cells contribute to the local signal.

The method is therefore delimited from higher-resolution methods which microscopically view the shape, division behavior and metabolism of individual tumor cells. These yield results that are of interest with regard to laboratory technology and for research, but they are suitable only to a very limited extent for a clinical application on the patient. The high resolution capability in the region of 1 μm that is necessary for the laboratory methods mentioned requires an epifluorescence microscope with high-aperture optical imaging which is not possible without immobilizing the patient. Furthermore, the tissue depth that can be achieved is distinctly limited. In order to obtain and evaluate the highly detailed information, comparatively long measurement times are required, for which reason such a method is unsuitable for clinical staging—unlike the method according to the invention.

The method according to the invention described above operates completely non-invasively. The skin surface is not touched, with the result that even in the case of a positive finding, there is no risk of the method initiating the flushing out of daughter cells of the tumor.

The combination of the fluorescence method with an OCT system yields a 3D-OCT owing to the three-dimensional scanning operation, such that the same tissue volume (“scanning volume”) is simultaneously assessed with regard to its malignant/benign differentiation with an expedient resolution, on the one hand, and is imaged morphologically, on the other hand. The examining physician therefore acquires a complete three-dimensional image of the tissue and its assessed metabolism state.

The method has been illustrated with regard to skin as a relatively easily accessible organ. However, it has significant potential for further development for other medical and laboratory-technological fields of application.

A further aspect of the invention specifies a method for visual characterization of tissue formed from cells, having the following steps:
providing a radiation source for emitting directional radiation;
irradiating the tissue to be characterized with the radiation at a measurement location, whereby a backscattered radiation which is characteristic of the tissue is excited in the tissue, wherein
the radiation emitted by the radiation source is periodically deflected transversely with respect to the propagation direction in such a way that the radiation periodically scans a region of the tissue around the measurement location which extends over a plurality of cells of the tissue.

Provision is often made for inferring the information concerning an object point from the integration of the fluorescence signal of a large number of excitation pulses. In order to obtain an excitation volume having approximately the same lateral extent from the elongated excitation focus, the beam is moved at high speed laterally in the x and y direction (beam axis is chosen in the z direction), whereby the fluorescence from a high number of excitation pulses can be utilized compactly with respect to an object point.

Since the frequency of the repetition rate of the laser is usually in the region of 80 MHz, conventional mechanical scanners are not appropriate as means for the “wobbling”.

Rather, a solution in the sense of microsystems engineering or micromechanics is resorted to here.

In a departure from the customary design of microscanners in which a uniaxially or biaxially cardanically suspended mirror is deflected in a controlled manner by means of an electromagnetic field counter to the restoring force of the leaf-spring-like carrier elements of the mirror, resonant guiding can be provided here. For this purpose, the mechanical resonant frequency of the mirror arrangement is determined by a suitable choice of the torsion spring moment and of the moment of inertia in such a way that the desired oscillation frequency arises. Precisely with this resonant frequency, excitation excitation is also effected by means of an electric or magnetic field. This gives rise to a sinusoidal oscillation of the mirror and corresponding movement of the focus in one axis. If “wobbling” is intended to be effected in two axes, then the resonant frequencies of the two mutually perpendicular oscillation axes are designed in different torsional and inertia moments, such that two different resonant frequencies arise in the two axes x and y. In this case, the low-frequency axis is designed in such a way that the oscillation takes place approximately 6 to 15 times more slowly than in the higher-frequency direction.

Consequently, the scanning spot moves with a Lissajous figure over the object field in the x and y direction and scans said field approximately uniformly. This resonant excitation in one axis or in two axes is referred to as “asynchronous biaxial harmonic driving” in the text below. Deflections using the Pockels or Kerr effect are possible as an alternative.

A further variant is the active phase modulation of a reflection with the aid of a micromechanical actuator. For this purpose, an array of freely oscillating mirrors which are each suspended in 3 or 4 lug points is produced micromechanically. The carrier strips of these individual and mutually identical phase actuators are embodied uniformly and have a small thickness in comparison with the width. As a result, they can readily be deflected perpendicular to the mirror surface without tilting, such that their position parallel to the rest position is maintained even in the case of deflection.

In the substrate layer below the mirror surfaces, an array of electrodes is provided in such a way that in the x-y direction per mirror element an electrode positioned underneath is present. The mirrors themselves likewise obtain a conductive contact area on the underside.

By driving the individual electrodes, an up and down movement of the mirror elements is achieved by means of electrostatic forces without torsion. As a result, an effective height position of each individual point can be driven in singular fashion. In the event of a reflection, this enables a phase shift which can be set in a location-dependent manner. The deflection of the mirrors only has to be 200 to 750 nm for optimal efficacy thereof at a customary wavelength in the visible region (400-1500 μm) for a complete phase extinction. This can be ensured without any problems by means of a suitable design of the carrier lugs.

What is essential with regard to the generation of partial beams is that in each partial beam the conditions are set in such a way that the desired intensity of the excitation radiation is achieved by means of the chosen optical system at the focus and that the partial beams are set in such a way that each of them forms, with the chosen optical focusing system, a focus separate from the foci of the other partial beams in the image plane of the optical system. Furthermore, the distance between these at least two foci in the image plane is set in such a way that their superimposition along the propagation direction of the excitation radiation, in particular in the vicinity of the focus of each partial beam, does not exceed a selectable fraction of the intensity of the excitation radiation at the focus.

To summarize, it is preferably provided that
the optical element for splitting into the desired number of partial beams is embodied as an actuating element in such a way that the number of partial beams and the distance between their foci can be chosen, that the partial beams or the overall beam can be modified in terms of their intensity by means of a suitable optical element in such a way that a suitable value can be set for the intensities at the focus of each partial beam in accordance with the requirements of the two- or multiphoton excitation process,
that the optical element for splitting into the desired number of partial beams or an additional optical element enables a time-variable deflection or arrangement of the partial beams which enables, in the image plane of the lens, a corresponding time-varied variation of the excitation region or parts thereof,
that, by means of such an arrangement, the resulting temporal variation of the excitation region is utilized for compensating for the gaps—resulting from the arrangement of the partial beams—of the excitation region in the focal region,
that the shaping of the excitation volume is obtained by “microwobbling”,
that the desired movements is obtained by mechanical or electromagnetic effects, such as Pockels or Kerr effect,
that micromechanical elements are used for rapid micromovement of the focus (or for phase control).

For reliable operation of an apparatus for visual examination of tissue there are the following fundamental requirements:
A. no harm whatsoever for the patient may occur in regular operation
B. likewise no harm whatsoever for the patient may occur in the “single-fault case”
C. the consequential effects are discussed for the worst case to be assumed from the combination of a plurality of malfunctions. Harm to the patient with pathological significance must be precluded.

The three requirements are assessed separately in the following sections as risk analysis according to ISO 14191. A subsequent conclusion summarizes the safety assessment.

### Description of Regular Operation (Case A): Categories:

In routine operation an apparatus according to the invention performs a scanning movement in that the focus of the laser is moved through the tissue laterally and in the depth and, by means of an intensity control, the attenuation of the beam in the tissue is compensated for and the intensity at the focus is kept constant. For safety reasons, it is necessary to meet the requirement that, on the one hand, an individual pulse does not cause any harm. On the other hand, the combined effect of the subsequent pulses and the energy accumulated during the application in the tissue must not lead to harm.

In the case of the individual pulse, it is necessary to consider adiabatic thermal effects, the influence of photochemistry and also intra- and intermolecular transition times on account of the fast energetic excitation processes. Since the highest excitation intensity lies at the focus of the laser radiation, the characteristic variables here are the volume density of the absorbed pulse energy and of the absorbed peak pulse power in the focus volume. Thermal conduction processes can be disregarded.

For the assessment of the combined effect of the subsequent pulses, the absorbed energy of the individual pulses should be related to the volume swept over by the scanning movement. For the case where the excitation volumes of the individual pulses overlap, a higher loading can occur here than as a result of an individual pulse.

While the absorption of the radiation in the focus volume is considered in the two cases above, the high degree of scattering of the tissue must be taken into account for evaluating the effect of the energy accumulated in the tissue. It has the effect that even in the wavelength range of the optical window in the case of comparatively low absorption, the radiation does not leave a limited tissue volume and is ultimately absorbed in said volume. What are characteristic here are the average power and also heat transfer processes in the tissue, that is to say that the thermal conduction and heat removal by blood circulation should be taken into account.

### Nonthermal Effects:

On account of the low photon energy (˜10⁴ cm⁻¹) and power density (˜10¹¹ W/cm²), damage caused by bond breaking can be ruled out. Damage caused by temperature gradients during heating in the ps time range are not known; therefore, the estimation of the tissue damage concentrates below on the purely thermal loading. Estimation and assessment of the thermal loading of the tissue:

Both the absorption and the scattering properties of the tissue have to be taken into account in the assessment of harmful side effects. In the envisaged wavelength range of the radiation, the effective cross section for scattering processes is one to two orders of magnitude higher than that for the absorption of the light. For 1064 nm, an absorption coefficient of 4 cm⁻¹ is specified for pure absorption and 5 cm⁻¹ as effective cross section for the combination of absorption and scattering. The penetration depth (decrease in intensity to 1/e) is estimated at 4 mm. Temperature changes arise on account of the short pulse duration (˜1 ps) initially adiabatically from the absorbed energy divided by the thermal capacity of the tissue. The lowest value for the thermal capacity (1930 J kg⁻¹ K⁻¹ for fatty tissue) is to be assumed here as a conservative estimation. The temperature differences then balance out again for relatively long times as a result of the thermal conductivity of the tissue. Here, too, the smallest value (λ=0.3 W m⁻¹ K⁻¹ in fat) is assumed for the estimation.

For the estimation of the adiabatic effect of an individual pulse it suffices to consider the location where the maximum intensity occurs. Said location lies at the focus, where the entire energy absorbed there during a laser pulse accumulates adiabatically, that is to say without exchanging heat with the surroundings. On account of the intensity control, this intensity is identical for all scanning positions, that is to say for all depths in the tissue. An energy transfer takes place only through linear and nonlinear absorption; scattering need not be taken into account in the calculation. Assuming an absorption of 50% per mm and the abovementioned thermal capacity, the apparatus parameters provided result in a heating of the focus volume by 2.6° C. by an individual laser pulse.

For the assessment of the combined effect of the subsequent pulses during the lateral scanning operation, it is necessary to take account of e.g. the absorbed energy of all the pulses which impinge on the tissue during 100 μm scanning section. This should be related to the corresponding scanning volume (width: waist diameter, depth: double Rayleigh length 100 μm). As a conservative estimation, this consideration is also to be carried out adiabatically, although a slight distribution of the input heat occurs in tissue in the time period between two pulses. The calculation using the abovementioned values results in a heating of the tissue by 4.7° C. in the focal region of the laser in the case of a single lateral sweep with the measurement spot. Since the scanning operation is effected “line by line”, that is to say that the measurement spot is moved laterally through the tissue first in a fixed depth before the next “line” is measured in a new depth, the combined effect of the pulses in the depth should not be estimated adiabatically. In the time period between two “lines”, the heat diffuses into a larger volume, such that the combined effect of two sagittally adjacent pulses is far exceeded by the joint effect of all the pulses that is considered in the following section.

For the assessment of this energy that has accumulated in the tissue on a long-term basis (that is to say over more than 10⁵ pulses), it should be related to the volume which is not left by the radiation on account of the high degree of scattering. The highest laser power, that is to say for the measurement at the greatest depth, is assumed as a conservative estimation. Assuming a cylindrical scattering volume with a diameter of 2 mm and a depth of 4 mm, the center of which is moved laterally rapidly on a line of 4 mm, heating by 1° C. per second arises in this volume. Under static conditions, that is to say if the applicator is not moved on the skin for a long time, the heat transfer of the tissue under worst-case conditions (fatty tissue, see above) limits the temperature to 43° C. in the beam plane (10 μm thickness). (Assumption: diffusive thermal conduction, at a distance of 5 mm the body temperature is kept at 37° C. by blood circulation).

In order to assess the significance of a temperature increase in the tissue from a technical safety standpoint, it is possible to have recourse to results in the literature (see, for example, G. Müller, H. P. Berlien “Angewandte Lasermedizin” [“Applied laser medicine”], ECOMED-Verlag, loose-leaf publication, 1993 ff., section 3.3 “Thermische Wirkungen” [“Thermal effects”]). Accordingly, a harmful thermal effect is dependent both on the temperature and on the action duration thereof. With the time scales considered here, the limit temperatures should be applied for less than 1 to at most 10 seconds. In accordance with the literature data, damage occurs with these action durations only at above 57° C., that is to say 20K above body temperature!

### Description of the Single-Fault Case (Case B):

the following fault sources should be considered for safety reasons:
a) the pulse energy increases to the maximum value of the laser due to a control fault
b) the scanning operation fails to occur, such that the subsequent pulses repeatedly strike the same location.

Fault treatment: as the most important measure against both the impermissible operating states a) and b), a fast electronic monitoring unit is provided in the construction and system layout and constantly checks the control operation and also the eye-safe contact of the applicator on the skin and, for its part, operates independently of the electronic control unit of the apparatus. As soon as the electronic monitoring unit identifies a fault, it switches the laser source off. The risk is thereby eliminated. As an additional measure, for case a), a hardware limitation that prevents an energy output beyond specific values is introduced into the laser.

### Assessment of the Residual Risk (Case C):

Probability of occurrence of the faults: the probability of occurrence of the abovementioned faults (pulse energy increases, scanning operation fails to occur) is lowered to an acceptable value by electronic measures and the independent electronic monitoring unit (precise definition can only be specified when the installation is established).

Consequential effect of the multi-fault case: should a fault nevertheless occur in the monitoring, then the effects should be evaluated as follows:

a) failure of the power control: the effect of the individual pulses and the combined effect of the subsequent pulses were calculated adiabatically for control operation. Therefore, the effect (disregarding nonlinear absorption) scales linearly with the laser power. The short-term adiabatic heating thus remains below 10° C. For the assessment of the energy accumulated in the tissue, as a rule the maximum laser power has already been reckoned with for the estimation. Consequently, a failure of the power control does not lead to harm to the patient.
b) failure of the scanning movement: the individual pulses are all focused in the same tissue volume, which leads to a rapid temperature increase until the focus volume reaches a constant temperature of 62° C. as a result of the thermal diffusion into the surrounding tissue. For the power accumulated in the tissue, a failure of the scanning movement means that a maximum temperature of 50° C. is established on the axis of the laser beam down to a depth of approximately 4 mm. Consequently, a failure of the scanning movement without the laser being switched off leads to the patient experiencing a pain stimulus. This tallies with self-experiments in which these radiation conditions were perceived as “pinpricks” on sensitive skin regions (elbow).

It should be noted that a failure of the scanning movement prevents the image generation and the treating physician therefore recognizes the disturbance. The laser will be immediately turned off even just by the applicator being lifted off the skin.

The invention is explained in more detail below on the basis of exemplary embodiments with reference to the figures, in which:

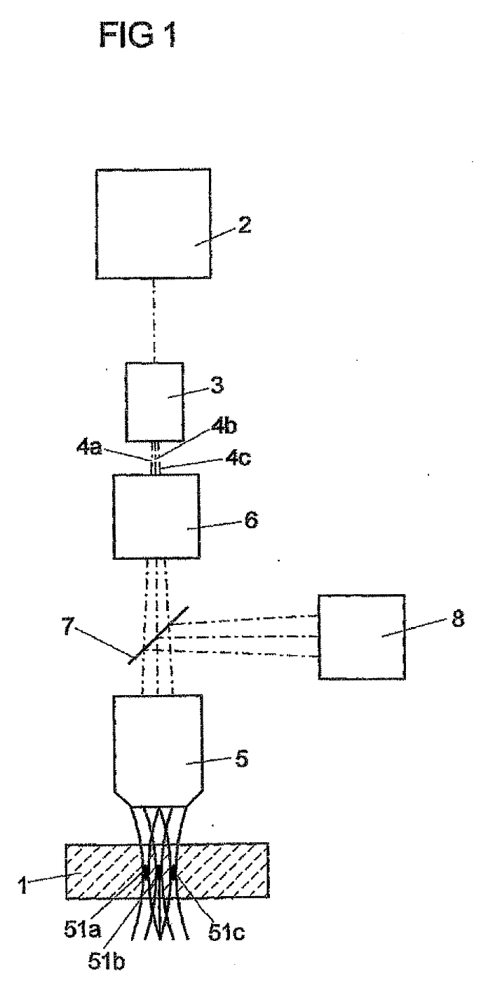

FIG. 1 schematically shows an apparatus for visual characterization of tissue in accordance with a first embodiment of the invention;

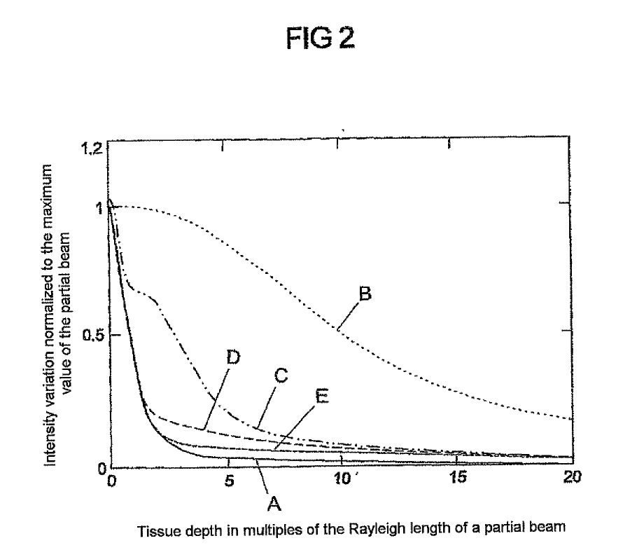

FIG. 2 shows by way of example the variation of the intensity for different intensity profiles;

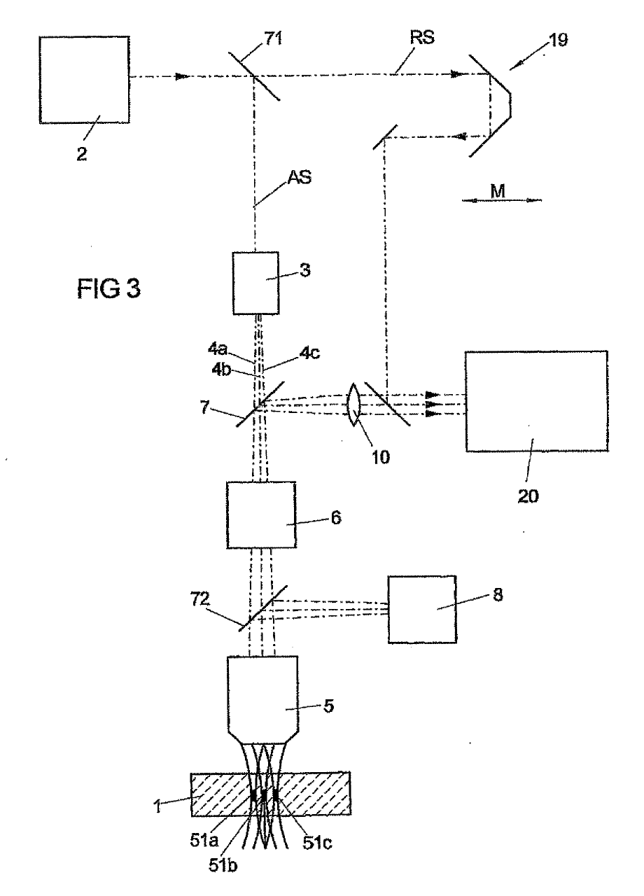

FIG. 3 schematically shows an apparatus for visual characterization of tissue in accordance with a second embodiment;

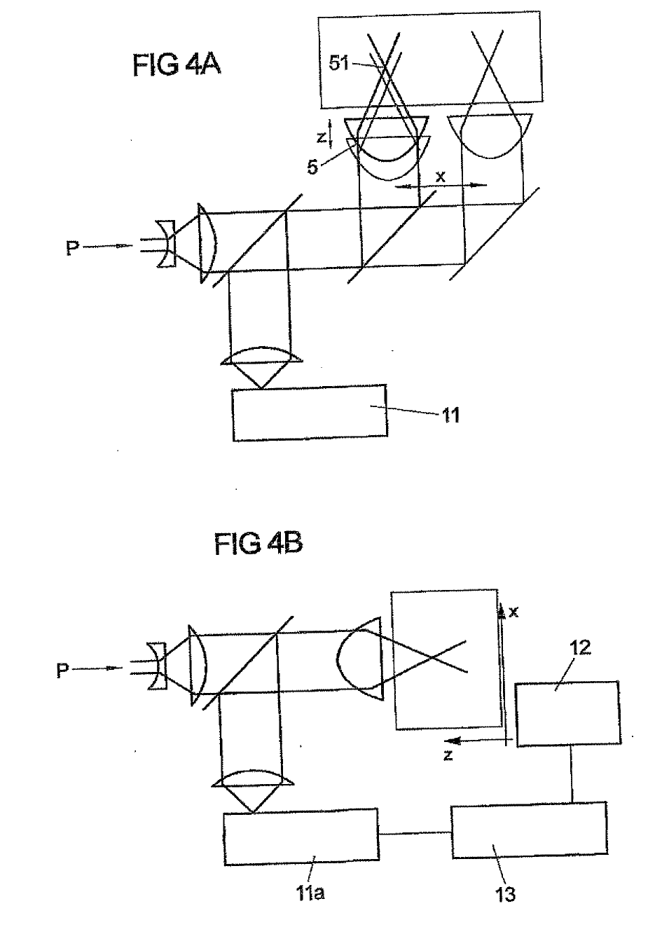

FIGS. 4A, 4B schematically show a third and fourth embodiment of an apparatus for visual characterization of tissue;

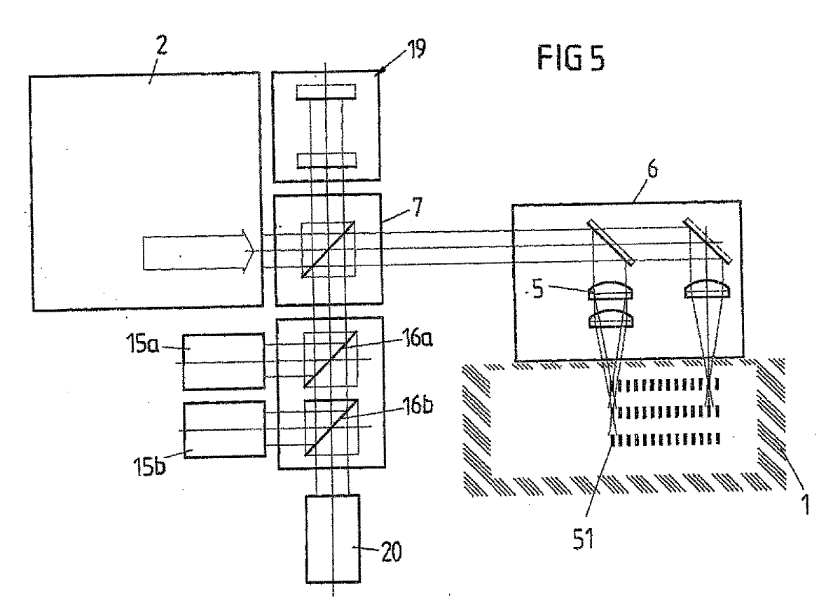

FIG. 5 shows a fifth embodiment of an apparatus for visual characterization of tissue;

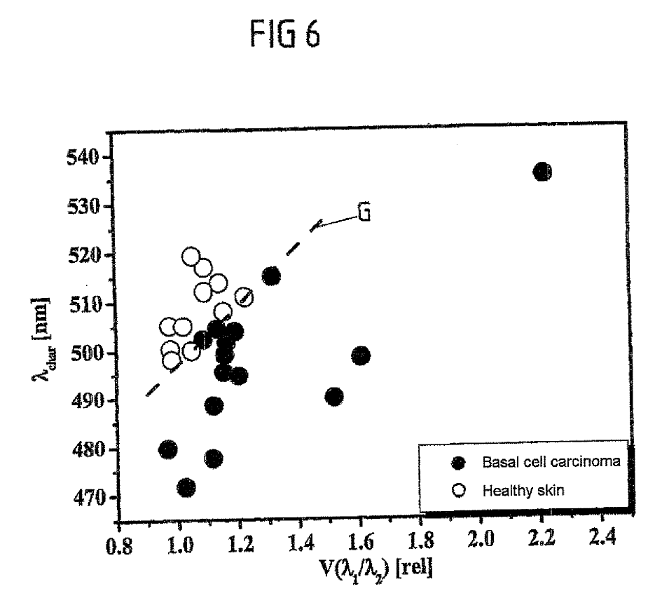

FIG. 6 shows a graphical illustration of a result of a fluorescence measurement;

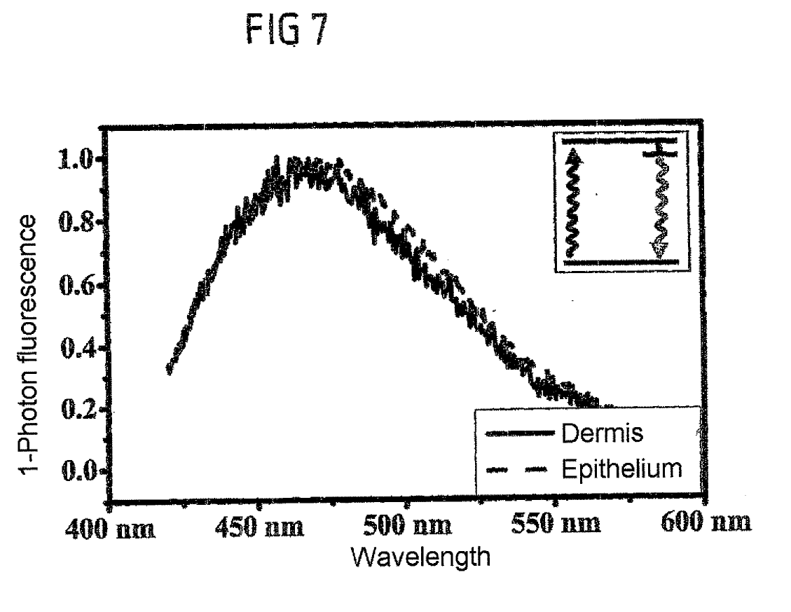

FIG. 7 shows a graphical illustration of the intensity of a single-photon fluorescence excited in a tissue;

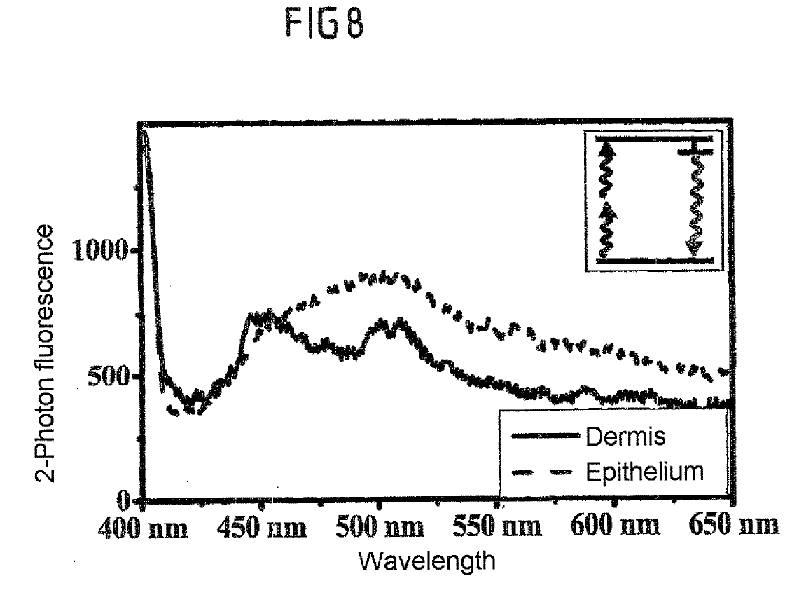

FIG. 8 shows a graphical representation of the intensity of a two-photon fluorescence excited in a tissue;

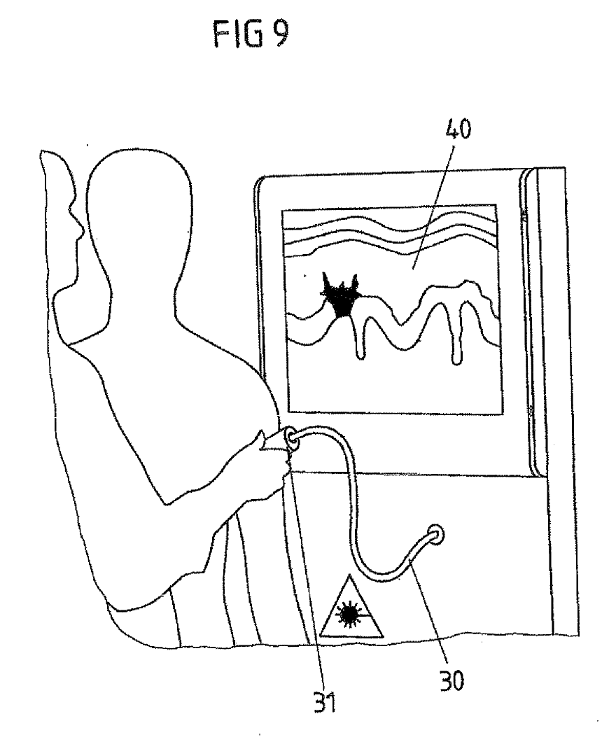

FIG. 9 shows a sixth embodiment of an apparatus for visual characterization of tissue.

FIG. 1 shows an apparatus for visual characterization of human or animal tissue 1. The light of a laser 2 that is typically used for initiating multiphoton processes is split into partial beams 4a, 4b, 4c by a beam splitter 3. Three partial beams are illustrated. It goes without saying, however, that the invention is not restricted to three partial beams, rather any desired number of partial beams can be used.

The different directions of said partial beams 4a, 4b, 4c are converted into a corresponding arrangement of foci 51a, 51b, 51c by means of a lens 5. On the optical path between beam splitting and lens 5, it is possible to introduce on the one hand elements for influencing e.g. the intensity of the partial beams (not illustrated here) or—as illustrated by way of example in FIG. 1—a scanner 6, which is used for the scanning excitation of the target 1 (tissue).

Multiphoton absorption takes place at the foci 51a, 51b, 51c of the partial beams 4a, 4b, 4c on account of the high intensities, as a consequence of which absorption these regions emit a corresponding fluorescence signal which can be used for characterization of the excitation region or excitation regions. This fluorescence light is partly detected by the lens 5, spectrally and spatially separated from the excitation light by a dichroic mirror 7 and detected by a fluorescence detector 8.

FIG. 1 makes it clear that the entire region (“integral excitation region”) which is covered by the bundle of excitation foci in the target is characterized by the summational detection of said fluorescence signal. Should it be necessary, for technical detection reasons, also to detect the gaps in this excitation pattern, then this can be done for example by minimal scanning movements and averaging over a plurality of laser pulses.

Such an arrangement is advantageous particularly when, for example in order to avoid damage, the intensity of the excitation radiation is upwardly limited. In the proposed arrangement, it is possible to apply the maximum permitted intensity to each partial beam. In accordance with the number of partial beams chosen, the fluorescence signal to be expected then increases in comparison with that from one partial beam. In this case however as described in the introduction—the spatial delimitation of the excitation region along the propagation direction of the excitation beam and thus the method resolution can be maintained largely unchanged.

The question—important according to the invention—of the suitable arrangement of the foci in the target is elucidated with reference to FIG. 2. FIG. 2 shows, for the selected case of the superimposition of nine partial beams of identical intensity, the dependence of the normalized intensity variations in the center of the bundle against the axis of the propagation direction of the radiation in multiples of the Rayleigh length of a partial beam. For the focal plane, z is set to zero.

In this case, the solid line A shows the variation for a partial beam. For comparison, the dotted line B shows the intensity variation for a beam having the same peak intensity as in A but—by means of a correspondingly chosen aperture—with the same total energy as the nine partial beams. This curve has a significantly slower subsidence of the intensity along the propagation direction. This means that the excitation region would be significantly lengthened on this axis.

The other three curves illustrate the intensity variation for different lateral distances between the foci of the partial beams (C: distance=1.5 times the waist radius; D: distance=triple the waist radius; E: distance=five times the waist radius).

If the distance is chosen to be too small (e.g. 1.5 times the waist radius), then the superimposition produces somewhat higher intensities at the focus itself and the excitation region is significantly enlarged along the propagation direction. This is evident if for example the point of subsidence to half the focus intensity is taken as a scale.

However, if for example triple or five times the waist radius is set in this exemplary arrangement, then the intensity has decreased by half approximately at the same distance from the focus as in the case of a beam having the same waist.

The example makes it clear that the advantageous choice according to the invention of the distance between the foci makes it possible to configure the extent of the excitation region along the propagation direction virtually independently of the number of partial beams and hence of the size of the excitation region.

The choice of the most favorable distance between the foci can be dependent on their arrangement in the focal plane of the lens, their number and the properties of the excitation radiation. A distance having the size of 2.5-15 times the waist radius of an individual beam is preferably chosen.

FIG. 3 shows an apparatus in which a laser beam source 2 is used both for the multiphoton excitation and for optical coherence reflectometry. If appropriate, the spectral properties of the source should be configured in such a way that it enables the desired depth resolution (resolution along the radiation propagation). Said radiation is split into an excitation beam AS and a reference beam RS by means of a partly transmissive mirror 71.

Both beams can additionally be provided with modulators which temporally modulate the intensity but not the optical path length. Such an arrangement is not represented.

A structure is impressed on the excitation beam AS transversely with respect to the propagation direction in the structuring unit 3, which structure emerges with high contrast on account of the focusing only in the vicinity of the focal plane of a lens 5 within the target. By way of example, a holographic element or microlens or microprism arrangements can be used for said unit. FIG. 3 illustrates, as a possible embodiment of such an arrangement, the splitting into three partial beams 4a, 4b, 4c that form closely adjacent foci in the target 1 (tissue). Outside the focal plane, the contrast between the foci decreases on account of the intermixing of the partial beams. The excitation beam AS structured in this way can be moved relative to the target 1 by means of a scanner 6 for the spatially defined excitation of said target.

Where the structured excitation beam AS illuminates the interior of the target 1, scattering takes place at structure boundaries. The backscattered portion passes through the lens 5, in which it is collimated again, and if appropriate the scanner 6, which reverses the scanning beam movement. By means of a beam splitter 7 and, if appropriate, a field lens 10, this light originating from the depth of the target 1 passes to interference with the reference beam RS in an interference detector 20 (e.g. a CCD camera).

The optical path length of the reference beam RS can be altered by means of a movable mirror 19 (the direction of movement is indicated by the double-headed arrow M). If the interference is observed in spatially resolved fashion, then a particularly high-contrast interference pattern will be observed if the optical path length of the reference beam corresponds precisely to the beam of the excitation light to the focus and of the backscattered light back to the superimposition location. The image of the structure impressed on the excitation beam will appear in the interference pattern. This is the indicator of the fact that the coherence reflectometry represents the focal region of the excitation.

As an example, two- or multiphoton microscopy is illustrated as a possible application in FIG. 3. In the example shown here, the multiphoton fluorescence is detected summationally from the focal regions of all three partial beams by means of the dichroic beam splitter 72 and the receiver 8. However, arrangements which permit the fluorescence signals of only a selected partial region to be detected are equally possible as well.

FIGS. 4A, 4B show the principle of a series apparatus for visual characterization of tissue. After the excitation by focused IR laser pulses (˜fs to ps) (arrow P), the light issuing from the sample/tissue is detected by a spectrometer 11. Said spectrometer can be embodied as a polychromator with a relatively low spectral resolution and wide spectral windows in order to simplify the evaluation and in order to improve the collection efficiency of the reflected light. In the latter case, the converging lens upstream of the polychromator 11 can also be emitted, if appropriate.

Since only light whose photons have a higher energy than that of the light radiated in is evaluated, it is ensured that the signal originates from nonlinear processes which can only take place in the region of the focus. The position of the focus 51 is unambiguously determined from the position of a lens 5 and the refractive index of the tissue. The depth assignment can be calibrated on a suitably prepared object.

The intensity is varied during the depth scan in order to compensate for the attenuation of the excitation light during focusing into deeper regions of the tissue. The control is based on physical effects which are initiated tissue-independently as a function of the excitation intensity at the focus and therefore also compensate for the attenuation in different types of tissue. Disturbing self-focusing and quantitative UV conversion in the tissue are avoided in this way.

Without restricting the generality, FIGS. 4A and 4B illustrate the relative movement between beam and focus in each case only in two coordinates (x lateral, z into the tissue depth). The second lateral axis y can additionally be followed according to the invention in one of the ways specified.

The principle of the arrangement of FIG. 4B corresponds to that of FIG. 4A, with the difference that the sample can be moved relative to the focusing lens by a positioning unit 12 in order to be able to measure different points of the sample. A spectrometer or polychromator (see corresponding note with regard to FIG. 4A) 11a is furthermore arranged, which enables temporally revolved measurements and is connected to an evaluation unit 13. The arrangement according to FIG. 4B is particularly suitable for in vitro examinations, such as e.g. cell cultures, of excised tissue samples or pathological preparations.

FIG. 5 shows a further variant of an apparatus for visual characterization of tissue with combined fluorescence and OCT system. The principle of this system has already been explained with reference to FIG. 3. The apparatus is not equipped with a spectrometer, but rather is operated with a plurality of discrete sensors 15a, 15b which utilize spectral partial signals of the fluorescence spectrum by filtering by means of filters 16a, 16b connected upstream.

The filters 16a/16b are not exclusively simply line, band or cut-off filters. Rather, at least in part complex graded filters are used which are calculated and produced in accordance with a weighting function.

By way of example, some typical operating values of such an apparatus shall be presented in tabular form below. In transferring the results from the preliminary experiments, with the following apparatus parameters it is possible to achieve a determination of the specific measurement results sought for the evaluation with a step size of 50 μm laterally and sagittally and an image repetition rate of 2 Hz on a slice of 1.5×4 mm²:

| Designation | | Value | | Note |
|---|---|---|---|---|
| Laser data: | | | | |
| Average power | P | 90 | mW | |
| Pulse duration | τ | 10 | ps | |
| Pulse repetition | fᵣₑₚ | 50 | kHz | |
| frequency | | | | |
| Beam data | | | | |
| Beam diameter at | D_{foc} | 10 | μm | |
| the focus | | | | |
| Rayleigh length | Z_{foc} | 80 | μm | = “depth of focus” |
| Effective volume | V_{foc} | 12 000 | (μm)³ | = π/4 * D_{foc}² * 2 Z_{foc} |
| Intensity at the | | 10¹¹ | W/cm² | Peak power at the |
| focus | | | | pulse maximum, |
| | | | | defined by intensity |
| | | | | control |
| Scanning data | | | | |
| Scanning frequency | f_{scan} | 5 | kHz | Averaging over 10 |
| | | | | laser pulses that |
| | | | | moved continuously |
| | | | | in the tissue. |
| Scanning step | Δx, | 50 | μm | = parallel to the |
| lateral | Δy | | | skin surface |
| Scanning step | Δz | 50 | μm | = into the tissue |
| axial | | | | depth |

The apparatus in accordance with FIG. 5 operates with nonlinear fluorescence excitation. This is for the following reasons:
A. A sufficient penetration depth is only possible within the “IR window” of approximately 750 to 900 nm.
B. As a result of the nonlinear effect, the fluorescence excitation occurs practically exclusively at the focus of the excitation, therefore in a highly localized manner, such that a high lateral and depth resolution are achieved.
C. The fluorescence spectra obtained by nonlinear excitation differ, according to our previous measurements, in principle from those which are obtained by means of linear excitation, that is to say at half the excitation wavelength.
The reason for this is that both the excitation kinetics and the emission kinetics and intramolecular energy transfer processes are essential to the fluorescence process. This insight is known in principle from relevant scientific investigations. The associated significantly improved specificity of nonlinear laser-spectroscopic methods is being employed beneficially in the meantime in some technical and biological-medical applications.

In the context of preliminary investigations with regard to the aforementioned point C, samples are used and examined in vitro which were defined histopathologically (basal cell carcinoma/skin). The new approach—nonlinear spectroscopy in supracellular average value measurement—enabled a very good distinguishability between basal cell carcinoma and normal tissue in the examinations. In the emission spectra thus generated, it was possible to identify spectral regions that practically permit a yes/no decision with regard to normal or anomalous cell metabolism. In a preliminary study, using this method a total of 26 series of measurements have been carried out and evaluated in the meantime. The result is illustrated in FIG. 6.

FIG. 6 shows an evaluation of the nonlinearly excited emission measurements on 26 samples, of which 11 represent healthy and 15 diseased tissue. The position of a characteristic wavelength (λ_{char}) calculated from the spectrum is plotted on the ordinate and the intensity ratio at two defined wavelengths V(λ₁/λ₂) is plotted on the abscissa. The interrupted line G represents a boundary curve. It can be discerned that basal cell carcinomas can be distinguished from normal skin.

It is evident that by suitable mathematical reduction of the measurement results, a self-referenced—i.e. independent of the total intensity—scalar characteristic variable can be determined, which shall be referred to hereinafter as Fluorescence-optical Tissue Indicator (FTI). The FTI at a specific geometrical measurement point in the tissue can be determined from the intensity values at some characteristic wavelengths. The valuation of these measurements shows that it is possible in a virtually unambiguous manner to distinguish diseased from healthy tissue by this nonlinear method. The precise method is the subject matter of the patent application.

The background for this new method is the fact that a series of molecular indicators are present in diseased tissue. However, said indicators cannot be distinguished by linear methods on account of their spectroscopic properties, as shown by FIG. 7.

FIG. 7 shows a conventional fluorescence-spectroscopic examination (UV-induced autofluorescence with single-photon excitation) of different types of tissue (dermis represented by a solid line and epithelial tissue represented by broken line). It can be discerned from the spectrum that no significant spectroscopic differences exist. This is not surprising since the substances considered in this context differ only very little in terms of their molecular structure. According to the invention, as a result of the wide absorption bands and the statistical averaging, no specificity should then be expected either.

A nonlinear method provides a remedy here in that the differences—significantly amplified under nonlinear conditions—between the different molecular systems can be evaluated spectroscopically. FIG. 8 illustrates how great these differences are. FIG. 8 shows the result of a nonlinearly excited spectroscopic examination of the different types of tissue. In contrast to FIG. 7, significant differences exist.

These few examples are intended to prove that the nonlinear spectroscopic methods open up a fundamentally new access to analysis precisely in the biological-medical field. These specific examples, without restricting the generality, show the principles of the novel methodology and prove the realizability thereof. The broader approach according to the invention is presented in the claims and explanations presented.

An essential part of the approach of the method according to the invention is that the FTI is detected with a spatial resolution that averages over the volume (focusing volume) of 20 to 200 000 cells. The fluorescence-optical tissue indicator is therefore a supracellularly averaged variable that describes the tissue, and not a characteristic variable for a cell organelle or specific cell compartments. It is only this definition that makes it possible, by data reduction to an extent appropriate for the diagnosis purpose, to perform a temporal evaluation in real time without fixing the patient over long periods of time.

The application of the FTI for the tissue measurements carried out up to then provides a clear demarcation between healthy and diseased tissue (see FIG. 4). To summarize, it can be established that the selectivity is obtained by the evaluation of the fluorescence data. The temporally and spatially congruent combination of these fluorescence data with an OCT permits the orientation of the physician in the tissue and supplies the assignment of the fluorescence-optical measurements to the morphology of the skin area examined.

The previous investigations show that a clear indicator of pathological states of the tissue could be found with the fluorescence tissue indicator FTI in conjunction with characteristic fluorescence maxima just in the case of pure intensity evaluation and ratio formation at two wavelengths (self-referenced).

Therefore, with the definition of the FTI set out by way of example, even now an indicator value which can clearly be demarcated has been found which enables an automatic differentiation between healthy and diseased by means of threshold values. The presented method principles according to the invention permit the selectivity and specificity to be increased further and to be specifically adapted to diverse issues.

Thus, according to the invention, in order to achieve an aim of diagnosis that is optimal and as meaningful as possible, with minimal false-negative and false-positive statements, and in order to increase the diagnosis statement, as explained, further wavelengths are used and/or a correlation factor is calculated for more than two wavelengths.

From the image point or focal point, the intensity values of the fluorescence radiation at two or more significant indicator or reference wavelengths can be determined and related by a ratio, It is thus possible to obtain the desired speed of evaluation and to afford the possibility of, in real time, both evaluating the measurement signals and making the good/poor decision, and of representing the result in the OCT image.

In this case, it is additionally possible to take account of
a) the decay behavior, that is to say the temporal component of the fluorescence emission, and
b) the influence of the fluorescence excitation wavelength.
Re a: By means of suitable fast sensors with a defined detection wavelength (for example PMT=photomultiplier tubes), the temporal variation can be evaluated by electronic gating.
Re b: By varying the excitation wavelength, the detection reliability and acuity are increased according to the invention.

Optional additional methods make it possible, in their combination with the new method described, to obtain additional information useful for the doctor.

This involves firstly the possible combination with an OCT method, which is not necessary for the specificity of the new method proposed, but provides the doctor with morphological additional information that makes it easier for him to perform the further treatment.

FIG. 9 shows a further embodiment of an apparatus for visual characterization of tissue. A laser source (not illustrated) is connected to a measuring head 31 via an optical fiber 30. The laser light can be passed to and away from the tissue to be examined by means of the optical fiber 30 and the measuring head 31. Consequently, in a manner similar to an ultrasound examination, a wide tissue area can be optically “scanned”. The backscattered light or light excited in the tissue is conducted via the optical fiber 30 into an integrated evaluation unit, which generates image information about the tissue from the data determined. Said image information is displayed on an integrated screen 40.

In addition to a fluorescence system it is possible to integrate an OCT system which operates synchronously and spatially congruently with the fluorescence system. This can be realized with relatively low apparatus outlay since the optical system, the scanner, the laser source and the signal path of the fluorescence system can be utilized.

Said OCT supplies, as important additional information, the morphological structure of those tissue volumes for which said information with respect to deviating metabolism is determined fluorescence-optically. This information can therefore be assigned directly to the morphological tissue image.

An image related to tissue structures is thus generated from the geometrical assignment to the measuring head. The physician acquires an indication of the tissue-morphologically characterized regions in the tissue in which the critical areas with conspicuous metabolism are situated.

The hallmark of the apparatus is a medical apparatus which is suitable for everyday practice or clinical routine and serves for efficient, reliable and reproducible tumor diagnosis. The effective principles, methods and algorithms which are used for this purpose are determined by the medical-diagnostic objective. This concerns for example the issues of single- or multicolor excitation and spectral and temporally resolved detection of the fluorescence. The dimensions of the diagnostic observation field follow from clinical practice: a depth resolution of 0.5-1.5 mm, sectional image representations having an edge length of a number of millimeters (e.g. 4 mm).

In this case, an essential characteristic of the diagnostic interpretation is the combination of the fluorescence-optical data with morphological information, because it is only by this means that, for example, the carcinogenic penetration of the basal membrane can be identified. According to the invention, the method of OCT or the extraction of corresponding fluorescence signals is used for such morphological information.

1-66. (canceled)

67. A method for visual characterization of human or animal tissue formed from cells, comprising the following steps:
providing a radiation source for emitting directional electromagnetic radiation; and
irradiating the tissue to be characterized with the radiation, whereby a reflection radiation which is characteristic of the tissue is generated in the tissue, wherein
the radiation that has penetrated into the tissue has, within an excitation region extending transversely with respect to the propagation direction of said radiation, a sufficient intensity to excite a characteristic reflection radiation in the tissue,
wherein
the radiation emitted by the radiation source has impressed on it an intensity profile transversely with respect to its propagation direction, said intensity profile being such that the excitation region covers a plurality of cells of the tissue and the excited reflection radiation originates from inter-cell tissue properties,
wherein impressing the intensity profile is effected by the radiation emitted by the radiation source being split into at least two partial beams, wherein the excitation region is defined by the position of the partial beams.

68. The method according to claim 67, wherein the partial beams are in each case focused into the tissue in such a way that the focal points for the partial beams are spaced apart from one another.

69. The method according to claim 68, wherein the intensity profile is chosen in such a way that the excitation region of an individual partial beam, transversely with respect to the propagation direction of the radiation issuing from the radiation source, extends over approximately 1 to 2,500 cells of the tissue.

70. The method according to claim 68, wherein the summation of the excitation region of the partial beams generated, transversely with respect to the propagation direction of the radiation issuing from the radiation source, extends over approximately 1,000 to 200,000 cells of the tissue.

71. The method according to claim 67, wherein the reflection radiation is a fluorescence signal, a harmonic of the radiation of the radiation source and/or a reflection signal.

72. The method according to claim 67, wherein the intensity of the radiation is chosen in such a way that two- or multiphoton excitation takes place in the tissue.

73. The method according to claim 67, wherein the backscattered radiation has double the frequency of the radiation with which the tissue is irradiated.

74. The method according to claim 67, wherein the radiation emitted by the radiation source, for additional characterization of the tissue by coherence reflectometry, is split into an excitation beam for exciting a fluorescence in the tissue and a reference beam, the excitation beam being partly reflected back from the tissue and the reference beam being superimposed with the radiation reflected back from the tissue.

75. The method according to claim 74, wherein the reference and excitation beams are superimposed in a spatially resolving interference detector.

76. The method according to claim 75, wherein the interference detector is a one- or two-dimensional image generator having at least 4 pixels, in particular a CCD chip, a CCD camera, a CCD line or a line camera.

77. The method according to claim 74, wherein the intensity profile impressed on the radiation issuing from the radiation source has a characteristic variation having at least two intensity maxima.

78. The method according to claim 75, wherein the optical path length for the reference beam is set in such a way that the intensity distribution impressed on the radiation emitted by the radiation source can be detected in the interference detector, wherein the optical path length for the reference beam corresponds to the optical path length covered by the excitation beam from the radiation source to the focus and back to the interference detector.

79. The method according to claim 78, wherein
a) the interference detector determines the intensity of the radiation arriving at it in a spatially resolved manner in a plane perpendicular to the radiation;
b) in each case the interference detector compares the intensities at adjacent locations with one another in order to determine a location-dependent contrast value; and
c) the interference detector determines from location-dependent contrast values an average value representing a characteristic integral contrast variable for the radiation arriving at the detector;
d) wherein the impressed intensity profile is detected by the integral contrast variable attaining a maximum.

80. The method according to claim 78, wherein
a) the interference detector determines the intensity of the radiation arriving at it in a spatially resolved manner in a detection plane perpendicular to the radiation;
b) a correlation value is determined from the intensity values determined and the intensity profile impressed onto the radiation of the radiation source;
c) the impressed intensity profile is detected by the correlation value attending a maximum.

81. The method according to claim 67, wherein the radiation emitted by the radiation source is deflected transversely with respect to its propagation direction such that a region of the tissue extending parallel to the tissue surface is scanned.

82. The method according to claim 67, wherein focusing of the radiation emitted by the radiation source into a focal plane is effected in a temporally dependent manner, such that the focal plane is displaced in the propagation direction of the radiation, whereby a region extending perpendicular to the tissue surface is scanned.

83. The method according to claim 67, wherein the backscattered radiation excited in the tissue is detected in a detector, its intensity being determined in a manner dependent on wavelength and/or time.

84. The method according to claim 67, wherein
a) the tissue is irradiated with radiation in the wavelength range of 720-800 nm, whereby a two-photon fluorescence is excited in the tissue;
b) the intensity of a first fluorescence signal at a wavelength of 460±30 nm and of a second fluorescence signal at a wavelength of 550±30 nm is detected;
c) the ratio of the intensities of the first and second fluorescence signals is determined; and
d) a signal is generated in a manner dependent on the ratio determined in step c).

85. The method according to claim 67, wherein
a) the tissue is irradiated with radiation in the wavelength range between 500-550 nm;
b) a first intensity of a two-photon fluorescence is detected at a wavelength of 340±40 nm;
c) the tissue is irradiated with radiation in the wavelength range between 720-800 nm;
d) a second intensity of a two-photon fluorescence is detected at a wavelength of 460±40 nm;
e) the ratio of the first intensity determined in step b) and the second intensity determined in step d) is determined; and
f) a signal is generated in a manner dependent on the ratio determined in step e).

86. The method according to claim 67, wherein
a) the tissue is irradiated with radiation in the wavelength range between 720-800 nm, and
b) an intensity of a two-photon fluorescence is detected at a wavelength of 460±40 nm.

87. The method according to claim 86, wherein the radiation is imaged into the tissue via a lens and the lens is displaced step by step along the propagation direction of the radiation emitted by the radiation source, steps a) and b) being effected for a plurality of lens positions, such that the focus is led in the propagation direction of the radiation through the tissue and fluorescence signal is determined for a plurality of tissue depths.

88. The method according to claim 87, wherein the method is carried out on a healthy tissue and the intensities of the fluorescence signal that are thus determined are stored as reference values.

89. The method according to claim 87 wherein
c) the method is carried out in accordance with claim 87 on a tissue to be characterized;
d) the intensities determined for each depth are related to the respective intensities of the stored reference signal by a ratio; and
e) a signal is generated in a manner dependent on the ratio determined in step d).

90. The method according to claim 89, wherein the signal is generated if the ratio determined in step d) deviates approximately 30% from the value 1.

91. The method according to claim 67, wherein
a) the tissue is irradiated with a short-pulse laser beam in the wavelength range of 720-880 nm or 980-1080 nm; and
b) the intensity of returning radiation at half the wavelength of the radiation used for irradiating the tissue in accordance with step a) is detected.

92. The method according to claim 91, wherein the radiation is imaged into the tissue via a lens and the lens is displaced step by step along the propagation direction of the radiation emitted by the radiation source, steps a) and b) being effected for a plurality of lens positions, such that the focus is led in the propagation direction of the radiation through the tissue and a fluorescence signal is determined for a plurality of tissue depths.

93. The method according to claim 92, wherein the method is carried out on a healthy tissue and the intensities of the fluorescence signal that are thus determined are stored as reference values.

94. The method according to claim 92, wherein:
c) the method is carried out in accordance with claim 91 on a tissue to be characterized,
d) the intensities determined for each depth are related to the respective intensities of the stored reference signal by a ratio; and
e) a signal is generated in a manner dependent on the ratio determined in step d).

95. The method according to claim 94, wherein the signal is generated if the ratio determined in step d) deviates approximately 40% from the value 1.

96. A method for visual characterization of tissue formed from cells, comprising the following steps:
providing a radiation source for emitting directional radiation; and
irradiating the tissue to be characterized with the radiation at a measurement location, whereby a backscattered radiation which is characteristic of the tissue is excited in the tissue, wherein the radiation emitted by the radiation source is periodically deflected transversely with respect to the propagation direction in such a way that the radiation periodically excites a region of the tissue around the measurement location which extends over a plurality of cells of the tissue.

97. The method according to claim 96, wherein the reflection radiation generated by the periodic excitation of the region around the measurement location is evaluated integrally.

98. The method according to claim 96, wherein the radiation is focused into the tissue, the intensity of the radiation being chosen in such a way that a characteristic reflection radiation is excited in a focal region extending around the focus along the propagation direction.

99. The method according to claim 98, wherein the focal region in which the characteristic reflection radiation is generated is periodically moved along the propagation direction of the radiation in order to scan a specific tissue volume around the measurement location.

100. The method according to claim 99, wherein the tissue volume has an extent transversely with respect to the propagation direction of the radiation of 2 to 10 mm.

101. The method according to claim 99, wherein the tissue volume has an extent in the propagation direction of the radiation of 0.3 to 4 mm.

102. The method according to claim 96, wherein the radiation source is a pulse source which emits pulsed radiation.

103. The method according to claim 102, wherein the pulse source has a pulse repetition frequency within the range of 35 to 150 MHz.

104. The method according to claim 96, wherein the periodic scanning around the measurement location is effected with a repetition frequency of 0.5 to 2 MHz.

105. The method according to claim 96, wherein the radiation issuing from the radiation source is deflected in addition to the periodic deflection which is effected around the measurement location transversely with respect to the propagation direction in order to scan the tissue at a plurality of measurement locations.

106. The method according to claim 105, wherein the periodic deflection around the measurement location is effected at a speed greater than the speed at which the radiation is additionally deflected in order to scan the tissue at a plurality of measurement locations.

107. The method according to claim 96, wherein the radiation emitted by the radiation source is split into at least two partial beams.

108. The method according to claim 107, wherein the partial beams are focused into the tissue.

109. The method according to claim 107, wherein the individual partial beams are deflected separately or jointly.

110. The method according to claim 107, wherein the partial beams are focused in such a way that the foci of adjacent partial beams are at a distance from one another, and the partial beams are deflected by a respective magnitude corresponding to said distance, such that gaps between the partial beams are compensated for.

111. An apparatus for characterizing tissue, comprising:
a radiation source for emitting directional radiation for irradiating the tissue to be characterized, which can excite in the tissue a backscattered radiation which is characteristic of the tissue,
radiation that has penetrated into the tissue having, within an excitation region extending transversely with respect to the propagation direction of said radiation, a sufficient intensity to excite a characteristic backscattered radiation in the tissue, and
beam shaping means with which the radiation emitted by the radiation source can have impressed on it an intensity profile such that the excitation region can cover a plurality of cells of the tissue and the backscattered radiation excited upon the irradiation of the tissue originates from inter-cell tissue properties,
wherein the beam shaping means are designed to split the radiation emitted by the radiation source into at least two partial beams.

112. The apparatus according to claim 111, further comprising imaging means for focusing the radiation emitted by the radiation source into the tissue.

113. The apparatus according to claim 112, wherein the imaging means are arranged upstream or downstream of the beam shaping means as seen from the radiation source.

114. The apparatus according to claim 112, wherein the imaging means comprise a plurality of lenses and each partial beam is focused into the tissue by a dedicated lens.

115. The apparatus according to claim 112, wherein the imaging means comprise at least one lens for focusing the radiation and also an adjusting unit, by which the at least one lens can be moved along the propagation direction of the radiation in order to move the focal point of the lens along said direction and to enable the tissue to be scanned in a plane extending perpendicular to the tissue surface.

116. The apparatus according to claim 115, wherein the adjusting unit and the lenses are integrated into a housing which can be placed onto the tissue to be examined and can be displaced parallel to the tissue surface.

117. The apparatus according to claim 116, further comprising electromotive and/or micromechanical actuating elements by which the housing can be displaced parallel to the tissue surface.

118. The apparatus according to claim 112, further comprising an optical fiber that leads the radiation generated by the radiation source to the tissue to be characterized.

119. The apparatus according to claim 118, wherein the optical fiber has the imaging means and/or the beam shaping means.

120. The apparatus according to claim 119, wherein the imaging means and/or the beam shaping means are formed integrally with the optical fiber.

121. The apparatus according to claim 111, further comprising a detector for detecting the backscattered radiation, which generates an electrical signal in a manner dependent on the detected radiation.

122. The apparatus according to claim 121, further comprising an evaluation unit for evaluating the electrical signal generated by the detector.

123. The apparatus according to claim 122, wherein the evaluation unit converts the electrical signal generated by the detector into an image information signal.

124. The apparatus according to claim 123, wherein the evaluation unit has a screen for visually displaying the image information signal.

125. An apparatus for characterizing tissue, comprising a radiation source for generating directional radiation for irradiating the tissue to be characterized and deflection means for deflecting radiation generated by the radiation source, by which the radiation can be periodically deflected transversely with respect to the propagation direction, such that the radiation can periodically excite a region of the tissue around a measurement location extending over a plurality of cells of the tissue.

126. The apparatus according to claim 125, wherein the deflection means comprise a mirror element for deflecting the radiation, which can be caused to effect a periodic movement by an electric or magnetic field.

127. The apparatus according to claim 126, wherein the mirror element has a first resonant frequency in a first direction transversely with respect to the propagation direction of the radiation, and has a second resonant frequency in a second direction, perpendicular to the first direction and to the propagation direction of the radiation.

128. The apparatus according to claim 125, wherein the radiation source is a pulsed laser.

129. The apparatus according to claim 128, wherein the laser emits radiation having a wavelength of between 500 and 1000 nm.

130. The apparatus according to claim 128, wherein the laser generates pulses having a pulse duration of between 80 fs and 800 ps.

131. The apparatus according to claim 125, further comprising a scanner for scanning the tissue at a plurality of measurement locations, wherein the scanner deflects the radiation issuing from the radiation source, in addition to the periodic deflection which is effected around the measurement location, transversely with respect to the propagation direction.

132. The apparatus according to claim 131, wherein the deflection means are designed to perform the periodic deflection around the measurement location at a speed greater than the speed at which the scanner additionally deflects the radiation in order to scan the tissue at a plurality of measurement locations.
